(19) United States
(12) Patent Application Publication
(10) Pub. No.: US 2004/0191855 A1
(43) Pub. Date: Sep. 30, 2004

(54) Production of secondary metabolites

(76) Inventors: Winston Daniel Leukes, Cape Town (ZA); Clive Garcin, Cape Town (ZA)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 10/478,899

(22) PCT Filed: May 17, 2002
(86) PCT No.: PCT/IB02/01686

(30) Foreign Application Priority Data
May 24, 2001 (ZA) 20014268

(51) Int. Cl. C12P 1/00; C12N 1/16

(52) U.S. Cl. 435/41; 435/254.3

(57) ABSTRACT

A method of producing a secondary metabolite includes providing an elongate porous substrate with a biofilm of micro-organism and which is arranged with one end of the substrate being at a higher elevation than the other end of the substrate so that the substrate is at an angle to the horizontal. A nutrient solution flows through the substrate, at a rate which is sufficiently low for a nutrient gradient to be established across the biofilm such that the nutrient concentration at a high level along the gradient is sufficiently high to support primary growth of the micro-organism, and the nutrient concentration at a low level along the gradient is sufficiently low to induce secondary growth of the micro-organism. The substrate's angle with the horizontal ensures that any droplets of nutrient permeate forming on the biofilm run towards the lower end of the substrate.

THIS INVENTION relates to the production of secondary metabolites. In particular, it relates to a method of producing a secondary metabolite, and to apparatus for producing a secondary metabolite.

Secondary metabolites are a group of compounds produced by a wide range of organisms as an adaptation to their natural environment. These compounds have found wide-spread application in the pharmaceutical and fine chemicals industries and are of considerable commercial interest.

Secondary metabolites in micro-organisms are produced in solid state culture as a result of differentiation and in liquid culture due to nutrient starvation. In the presence of a nutrient solution of sufficiently high concentration, most micro-organisms exhibit exponential growth, referred to as primary growth. As the concentration of the nutrient solution falls, the micro-organisms, in response to the stress caused by nutrient starvation, adapt and switch to what is referred to as secondary metabolism in which they start to produce the secondary metabolites. Typically, in commercial applications using conventional technology, secondary metabolites are produced in batch culture.

As mentioned above, many of the secondary metabolites are of considerable commercial interest as they have useful properties. Phanerochaete chrysosporium, for example, is a filamentous fungus capable of degrading a wide range of recalcitrant aromatic pollutants. These compounds include BTEX (Benzene, Toluene, Ethylbenzene and Xylene) type compounds, DDT, TCDD (2, 3, 7, 8-tetrachlorodibenzo-p-dioxin), benzo(a)pyrene, Lindane and certain PCB congeners. This organism has thus been considered a candidate for the bioremediation of waste waters containing such pollutants.

This degradative ability is due in part to the secretion, during stationary or secondary metabolism phase initiated by nutrient limiting conditions, of a group of H₂O₂-producing oxidases as well as a group of peroxidases including lignin peroxidase (LiP) and manganese peroxidase (MnP). In whole cell cultures, however, a certain amount of biodegradation of these compounds occurs independently of the secretion of these enzymes.

The Applicant is aware of technology in which fungal biofilms are immobilised on hollow fibre ultrafiltration membranes for the purpose of producing secondary metabolites of commercial interest. The technology to date has used horizontally orientated fibres and the Applicant has found that this technology has certain drawbacks, including that biofilm growth is inconsistent along the fibre length; permeate droplets form on the biofilm, which leads to nutrient localisation and hence excessive growth in some parts and poor growth in others; in multi-fibre systems, permeate droplets from upper fibres fall onto and interfere with the biofilms on lower fibres; and it is difficult to characterise and model such inconsistent biofilms, which make it difficult to produce commercially viable systems. Furthermore, the hollow fibre ultrafiltration membranes do not have a uniform permeability along their length because of a non-uniform pore size distribution and other manufacturing inconsistencies. This compounds the problem of inconsistent biofilm growth and droplet formation, as the nutrient flux can vary up to one order of magnitude along the membrane length.

It is an object of the present invention to provide a method and apparatus for the production of secondary metabolites which can operate on a continuous basis and which at least alleviates the problems of the prior art.

According to one aspect of the invention, there is provided a method of producing a secondary metabolite, which method includes

providing an elongate porous substrate which has a biofilm of micro-organism attached thereto and which is arranged with one end of the substrate being at a higher elevation than the other end of the substrate so that the substrate is at an angle to the horizontal; and

causing a nutrient solution to flow through the substrate, at a rate which is sufficiently low for a nutrient gradient to be established across the biofilm such that the nutrient concentration at a high level along the gradient is sufficiently high to support primary growth of the micro-organism, and the nutrient concentration at a low level along the gradient is sufficiently low to induce secondary metabolism of the micro-organism, thereby to produce a secondary metabolite, the angle with the horizontal at which the substrate is arranged being sufficient to ensure that any droplets of nutrient permeate forming on the biofilm run towards the lower end of the substrate.

Preferably, the angle with the horizontal at which the substrate is arranged is substantially 90°. This advantageously ensures, when multiple spaced substrates arranged parallel to each other are used, that droplets from one substrate do not drip onto another substrate.

An outside or exposed surface of the biofilm remote from the substrate may be contacted with an oxygen-containing gas to provide oxygen for metabolism. The oxygen-containing gas may be air.

The oxygen-containing gas may be blown over the outside surface of the biofilm, to carry away spores and dead cells of the micro-organism.

The micro-organism may be a filamentous fungus. The filamentous fungus may be Phanerochaete chrysosporium.

According to another aspect of the invention, there is provided apparatus for producing a secondary metabolite, which apparatus includes

at least one elongate porous substrate having two opposed surfaces and which is arranged with one end of the substrate being at a higher elevation than the other end of the substrate so that the substrate is at an angle to the horizontal; and

a feed arrangement for feeding a nutrient feed solution for micro-organisms into contact with one surface of the substrate so that the nutrient feed solution can permeate through the substrate to the other surface of the substrate, which is a biofilm-coated surface in use, the angle with the horizontal at which the substrate is arranged being sufficient to ensure that any droplets of nutrient permeate forming on the biofilm run toward the lower end of the substrate.

The feed arrangement may be configured to feed the nutrient feed solution at a high or a low elevation into contact with the one surface of the substrate, e.g. at an upper end of the substrate.

The apparatus may include a discharge arrangement for removing nutrient feed solution from the one surface of the substrate. The discharge arrangement may be configured to remove the nutrient feed solution at a low or a high elevation from the one surface of the substrate.

The apparatus may include a housing for the porous substrate, the housing being spaced from the porous substrate. The housing may include a gas inlet for feeding a gas into contact with the other or biofilm-coated surface of the substrate, and an outlet for discharging gas and/or permeate from the housing.

Typically, the gas inlet is at a high elevation, e.g. at substantially the same elevation as the upper end of the porous substrate, and the outlet is at a low elevation, e.g. at or below a lower end of the porous substrate.

The substrate may be in the form of a hollow fibre membrane, with the outside of the membrane being in use the biofilm-coated surface.

The hollow fibre membrane may have a relatively thin, porous skin on the inside, and a relatively thick, finger-like, externally unskinned void structure radiating outwardly from the skin. It may have an outside diameter of about 2 mm, a porous skin having a thickness of about 1 μm and a void structure having a thickness of about 300 μm.

Preferably, the apparatus includes a plurality of elongate porous substrates, e.g. a plurality of hollow fibre membranes, spaced from each other and arranged at a substantially 90° angle to the horizontal.

The invention will now be described, by way of example, with reference to the accompanying diagrammatic drawings and the Examples.

In the drawings,

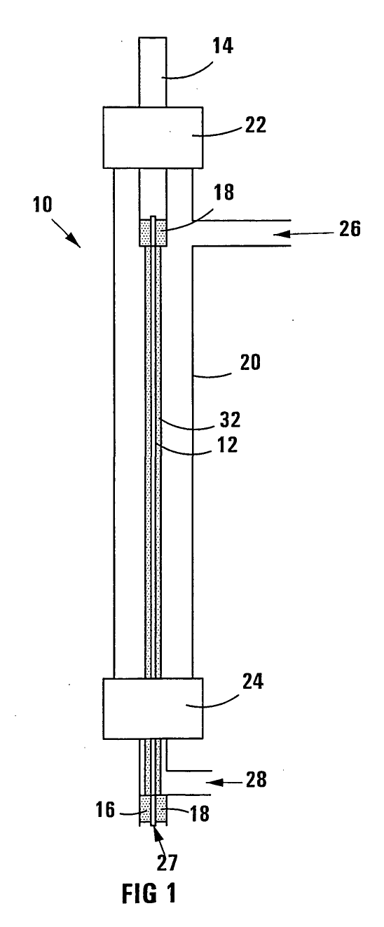

FIG. 1 shows an elevational side view of one embodiment of apparatus in accordance with the invention for producing a secondary metabolite;

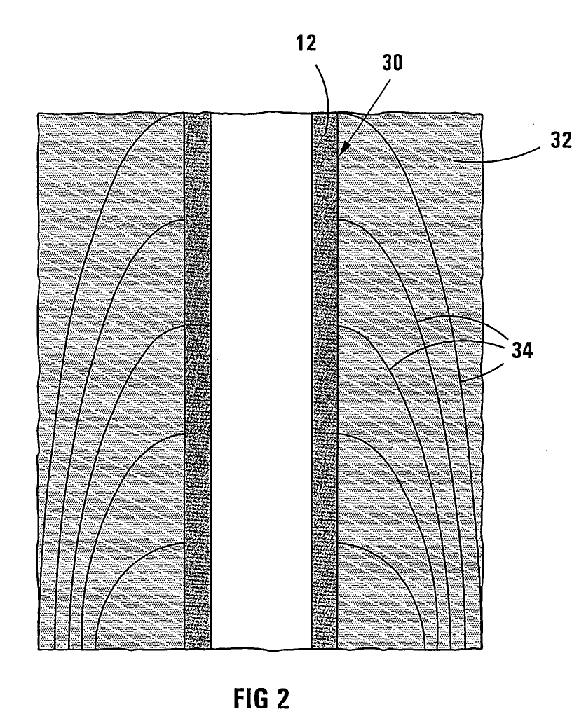

FIG. 2 shows an enlarged sectional view of a portion of a porous substrate of the apparatus of FIG. 1, coated on one side thereof with a biofilm, and illustrates a nutrient solution flow regime through the biofilm; and

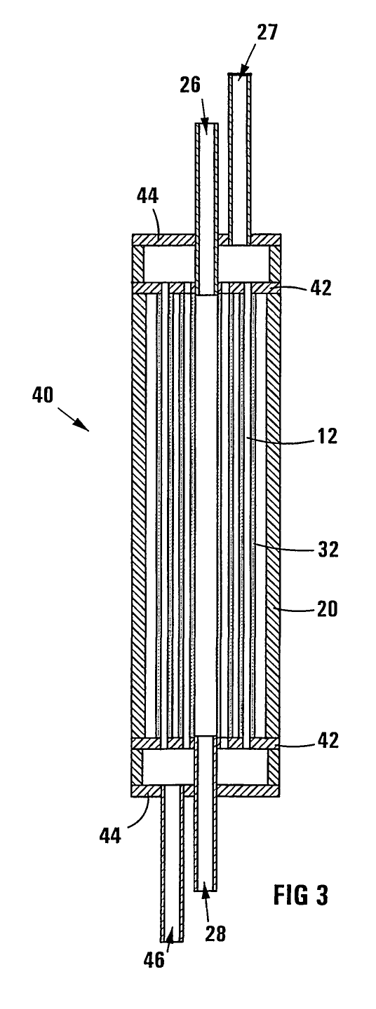

FIG. 3 shows an elevational side view of another embodiment of apparatus in accordance with the invention for producing a secondary metabolite.

Referring to FIG. 1 of the drawings, reference numeral 10 generally indicates apparatus in accordance with the invention for producing a secondary metabolite. Although the apparatus or bioreactor 10 shown in FIG. 1 of the drawings is at a laboratory scale, it is to be appreciated that the principles embodied in the apparatus of FIG. 1 can easily be applied to an up-scaled or commercial embodiment.

The bioreactor 10 includes an externally-unskinned polysulphone hollow fibre capillary membrane 12 with ends of the membrane being potted into glass inserts 14, 16 with epoxy 18. A housing or reactor shell 20 of glass is arranged coaxially with the capillary membrane 12 and is provided with end caps 22, 24 which screw onto the glass housing 20. The housing 20 defines a gas inlet 26. The glass insert 14 defines a feed arrangement for feeding a nutrient feed solution for micro-organisms into the lumen of the hollow fibre capillary membrane 12. A nutrient solution outlet from the lumen is provided at 27.

The glass insert 16 defines an outlet 28 for the housing 20 for discharging gas and permeate from the housing 20.

In use, a biofilm 32 is established on an external surface 30 (see FIG. 2) of the capillary membrane 12. This is achieved by reverse filtering a spore or vegetative inoculum of the desired micro-organism through the capillary membrane 12 and draining any permeate out the lumen through the outlet 27. The inoculum is thus immobilised on the membrane surface 30.

An appropriate nutrient solution for the micro-organism is then supplied from above via the glass insert 14 so as to perfuse the lumen continuously, but at a rate sufficient to allow gradients of the growth limiting nutrient to occur in the biofilm 32 established on the surface 30. The nutrient feed solution exiting through the outlet 27 is pumped back to the glass insert 14 to be recycled through the lumen of the capillary member 12. Some of the nutrient feed solution permeates through the capillary membrane 12 forming permeate droplets on the biofilm 32 and run down the biofilm 32. Humidified air is fed into the housing 20 by means of the gas inlet 26 and vented through the outlet 28. The secondary metabolite is collected in the nutrient feed solution permeate which is also removed through the outlet 28.

Fundamental to the production of secondary metabolites is the concept of nutrient starvation, which stresses the micro-organism and thus encourages metabolite production. This is achieved with the membrane-immobilised biofilm bioreactor 10 by the production of radial nutrient concentration gradients through the biofilm 32. Thus, the nutrient concentration at the membrane/biofilm interface is high, whereas at the outer edge or exposed surface of the biofilm 32 the nutrient concentration is low, with the reverse being true for oxygen which diffuses into the biofilm 32 from the air fed into the housing 20. Secondary metabolites are continuously produced at the biofilm outer edge due to secondary metabolism of the micro-organisms and a continuous biofilm population primary growth at the membrane/biofilm interface is achieved, due to biofilm differentiation. As new biomass is laid down, older cells are displaced outward until they are shed from the outside surface of the biofilm 32. As the cells move from the inside of the biofilm to the outside they move from an environment that is nutrient-rich and thus supports primary growth, to an environment that is nutrient-poor and causes the micro-organism to switch to secondary metabolism and thus leads to the production of secondary metabolites. The process is stable and steady-state, and can thus be operated on a continuous basis. Also, the thickness of the biofilm 32 and immobilisation of the organism may contribute to the rate of secondary metabolite production being high.

The air that is blown through the bioreactor shell 20 serves to supply the oxygen that is required for viability of the biofilm, and also to carry away spores and dead cells that are shed from the outer surface of the biofilm 32.

As mentioned hereinbefore, during operation of the bioreactor 10, nutrient feed solution permeate forms droplets on the biofilm 32, which droplets run down the biofilm 32 to the glass insert 16. This is in contrast with multi fibre prior art systems, in which permeate droplets from upper fibres fall onto and interfere with the biofilms on lower fibres.

In addition to managing the removal of permeate droplets, the vertical arrangement of the capillary membrane 12 also ensures axial nutrient gradients in the biofilm 32, in addition to the radial nutrient gradients. This is as a result of the unique gravity affected flow regime of nutrient solution through the biofilm 32, as clearly illustrated in FIG. 2 of the drawings. In FIG. 2, the lines 34 illustrate in two dimensions the nutrient flow regime through the biofilm 32. The bioreactor 10 thus advantageously resembles the natural environment of micro-organisms by providing a solid/liquid gas interface typical of solid state culture while offering continuous perfusion of liquid nutrients similar to liquid culture to achieve high productivity.

Referring to FIG. 3 of the drawings, reference numeral 40 generally indicates another embodiment of an apparatus or bioreactor in accordance with the invention for producing a secondary metabolite. The bioreactor 40 is similar to the bioreactor 10, and unless otherwise indicated, the same reference numerals are used to indicate the same or similar parts or features.

The bioreactor 40 resembles in some respects a shell and tube heat exchanger and includes, unlike the bioreactor 10, a plurality of vertically arranged, externally-unskinned polysulphone hollow fibre capillary membranes 12. The outside diameter of each capillary membrane 12 is 2 mm and the ends of each capillary membrane 12 are potted in an end plate 42. The capillary membranes 12 are equally spaced in a hexagonal close packing arrangement. The shell 20 is defined by a translucent PVC-tube, the ends of which are closed by the end plates 42. A head 44 is provided at a bottom and upper end of the shell 20. The different components of the bioreactor 40 are held together with epoxy glue. The gas inlet 26 and the nutrient solution outlet 27 are provided at the upper end, with the gas inlet 26 protruding through the upper head 44 and extending through the upper end plate 42 and the nutrient solution outlet 27 extending through the upper head 44 only.

The outlet 28 for gas and permeate extends through the lower end plate 42 and protrudes through the lower head 44. A nutrient solution inlet or feed arrangement 46 extends through the lower head 44.

Further details of the bioreactor 40 are provided in Table 1:

TABLE 1

| | | |
|---|---|---|
| Reactor Diameter | 27 | mm |
| Reactor Volume | 0.19 | L |
| Active Membrane Length | 333 | mmm |
| Number of Membranes | 12 | |
| Membrane Surface Area | 1,506 × 10⁻² | m² |

Before use, the bioreactor 40 is sterilized with a 4% formaldehyde solution and then rinsed with sterile water. Thereafter it is inoculated either with a spore suspension or with a homogenised vegetative inoculum by reverse filtration of the inoculum so that it is immobilized on the outside surfaces of the capillary membranes 12, as hereinbefore described, to establish a biofilm 32 on the external surface of each capillary membrane 12.

The bioreactor 40 is used in similar fashion as the bioreactor 10. Thus, an appropriate nutrient solution for the micro-organism immobilized on the capillary membranes 12 is supplied from a reservoir via a peristaltic pump (not shown), through the inlet 46. The nutrient solution perfuses the lumen of each capillary membrane 12 as hereinbefore described with reference to the bioreactor 10, before exiting through the outlet 27.

Humidified oxygen or air is supplied to the extra-capillary space inside the shell 20 through the inlet 26. The oxygen or air leaves the bioreactor 40 through the outlet 28.

Some of the nutrient feed solution permeates through the capillary membranes 12 forming permeate droplets on the biofilms 32 and run down the biofilms 32 onto the lower end plate 42. The permeate, together with any secondary metabolite produced by the biofilm 32 is removed through the outlet 28.

During operation of the bioreactor 40, air is changed for pure oxygen periodically to stimulate secondary metabolite production.

### EXAMPLE 1

The bioreactor 10 of FIG. 1 was used to produce magnesium peroxidase as a secondary metabolite from a biofilm of Phanerochaete chrysosporium. Table 2 provides the operational parameters and results of the experiment:

TABLE 2

| | |
|---|---|
| Organism | Phanerochaete chrysosporium BKM-F |
| | 1767 |
| Nutrient feed medium | According to: Tien, M and Kirk, TK, |
| | Lignin peroxidase of Phanerochaete |
| | chrysosporium. Methods Enzymology |
| | 161: 238-248 |
| Transmembrane nutrient flux | 0.2-3 L · m⁻² · hr⁻¹ |
| Active membrane length | 170 mm |
| Membrane diameters | 1.4 mm ID, 1.9 mm OD |
| Glass module diameter | 20 mm |
| Air flow rate | ˜20 L · hr⁻¹ |
| Secondary Metabolite | Manganese peroxidase |
| produced | |
| Peak productivity | *304.5 Units · L⁻¹ (reactor volume) day⁻¹ |
| Peak concentration | *354.9 Units · L⁻¹ |
| Final biomass density | ˜14 mg · cm⁻² (membrane area) |
| Experimental run time | 300 hrs |

*MnP activity determined using ABTS as a substrate.

### EXAMPLE 2

Phanerochaete chrysosporium strain BKM-F 176-7 was used as test organism in the bioreactor 40. Manganese peroxidase (MnP), an enzyme produced during secondary metabolism, was measured in the product by assaying according to M.del Pilar Castillo, J. Stenstrom and P. Ander (1994). Determination of Manganese Peroxidase Activity with 3-Methyl-2-benzothialinone Hydrazone and 3-(Dimethylamino) benzoic acid, Analytical Biochemistry 218, 399-404. Air flow through the bioreactor 40 was measured with a tapered wall rotameter. Reactor internal pressure and transmembrane pressure were measured with a mercury manometer. Transmembrane flux was calculated by dividing the permeate (product) flow rate by the membrane surface area. MnP concentration in the permeate was reported as Units per liter of permeate where one unit is defined as 1 μmol of enzyme substrate converted in one minute. Productivity of the reactor is reported as units of enzyme produced per liter reactor volume per day. Table 3 lists some results for enzyme production.

TABLE 3

| | Flow | | Enzyme | | |
|---|---|---|---|---|---|
| Time | Rate | Flux | Conc. | Productivity | |
| (hrs) | (ml/hr) | (Lm⁻² · hr⁻¹) | (U/L) | (U · L⁻¹ · day⁻¹) | Comment |
| 62 | 19 | 1.25 | 0 | 0 | |
| 86 | 1.3 | 0.089 | 71 | 12 | |
| 110 | 0.58 | 0.04 | 556 | 41 | |
| 134 | 5.1 | 0.34 | 1221 | 785 | |
| 158 | 4.3 | 0.29 | 1967 | 1066 | |
| 182 | 4.7 | 0.31 | 1500 | 888 | |
| 206 | 4.3 | 0.29 | 2434 | 1319 | |
| 230 | 3.7 | 0.24 | 2281 | 1063 | |
| 254 | 4.7 | 0.31 | 968 | 573 | |
| 280 | 4.5 | 0.3 | 1714 | 972 | Switch to |
| | | | | | Oxygen |
| 300 | 2.1 | 0.14 | 16171 | 4279 | |
| 324 | 1 | 0.067 | 3494 | 440 | Switch back |
| | | | | | to Air |

The method and vertical bioreactor of the invention, as illustrated, overcome the problem of membrane inconsistency and droplet formation experienced with prior art technology, resulting in a more uniform delivery of nutrients to the biofilm. This in turn results in a more homogenous biofilm, which has important implications for scale-up systems, in that biofilms are less likely to breach adjacent membranes and thus cause clogging of the reactor, which decreases oxygen mass transfer and thus productivity. Furthermore, the vertically orientated porous substrate more closely approaches the desired radial nutrient concentration gradient through the biofilm around the capillary membrane at any particular point along the length of the capillary membrane than does a horizontal porous substrate. Accordingly, higher secondary metabolite production is possible. In fact, the vertical bioreactor of the invention, as illustrated, provides much higher productivity and yield compared to the bioreactors of the prior art. Advantageously, the bioreactor of the invention, as illustrated, can be used on a continuous basis.

1 A method of producing a secondary metabolite, which method includes
providing an elongate porous substrate which has a biofilm of micro-organism attached thereto and which is arranged with one end of the substrate being at a higher elevation than the other end of the substrate so that the substrate is at an angle to the horizontal;
causing a nutrient solution to flow through the substrate, at a rate which is sufficiently low for a nutrient gradient to be established across the biofilm such that the nutrient concentration at a high level along the gradient is sufficiently high to support primary growth of the micro-organism, and the nutrient concentration at a low level along the gradient is sufficiently low to induce secondary metabolism of the micro-organism, thereby to produce a secondary metabolite, the angle with the horizontal at which the substrate is arranged being sufficient to ensure that any droplets of nutrient permeate forming on the biofilm run towards the lower end of the substrate; and
removing the nutrient permeate, which includes the secondary metabolite, from the lower end of the substrate.

2 A method as set forth in claim 1, in which the angle with the horizontal at which the substrate is arranged is substantially 90°.

3 A method as set forth in claim 1 in which an outside or exposed surface of the biofilm remote from the substrate is contacted with an oxygen-containing gas to provide oxygen for metabolism.

4 A method as set forth in claim 3, in which the oxygen-containing gas is blown over the outside surface of the biofilm, to carry away spores and dead cells of the micro-organism.

5 A method as set forth in claim 1, in which the micro-organism is a filamentous fungus.

6 Apparatus for producing a secondary metabolite, which apparatus includes
at least one elongate porous substrate having two opposed surfaces, one of which is in use a nutrient-contacting surface and one of which is in use a biofilm-coated surface, the substrate being arranged with one end of the substrate being at a higher elevation than the other end of the substrate so that the substrate is at an angle to the horizontal, and the nutrient-contacting surface and the biofilm-coated surface, although being sealed from each other, allowing nutrient permeation to take place through the porous substrate from the nutrient-contacting surface to the biofilm-coated surface;
a feed arrangement for feeding a nutrient feed solution for micro-organisms into contact with the nutrient-contacting surface of the substrate so that the nutrient feed solution can permeate through the substrate to the biofilm-coated surface in use, the angle with the horizontal at which the substrate is arranged being sufficient to ensure that any droplets of nutrient permeate forming on the biofilm run toward the lower end of the substrate; and
a permeate outlet to remove nutrient permeate from the lower end of the substrate.

7 Apparatus as set forth in claim 6, in which the feed arrangement is configured to feed the nutrient feed solution at a low elevation into contact with the nutrient-contacting surface of the substrate.

8 Apparatus as set forth in claim 6 which includes a discharge arrangement for removing nutrient feed solution from the nutrient contacting surface of the substrate, the discharge arrangement being configured to remove the nutrient feed solution at a high elevation from the nutrient-contacting surface of the substrate.

9 Apparatus as set forth in claim 6, which includes a housing for the porous substrate, the housing being spaced from the porous substrate and including a gas inlet for feeding a gas into contact with the biofilm-coated surface of the substrate, and an outlet for discharging gas from the housing.

10 Apparatus as set forth in claim 6, in which the substrate is in the form of a hollow fibre membrane, with the outside of the membrane being in use the biofilm-coated surface.

11 Apparatus as set forth in claim 10, in which the substrate is in the form of a plurality of said hollow fibre membranes spaced from each other and arranged at a substantially 90° angle to the horizontal.
